(19) United States
(12) Patent Application Publication
(10) Pub. No.: US 2018/0315502 A1
(43) Pub. Date: Nov. 1, 2018

(54) METHOD AND APPARATUS FOR OPTIMIZATION AND SIMULATION OF PATIENT FLOW

(71) Applicant: KONINKLIJKE PHILIPS N.V., Eindhoven (NL)

(72) Inventors: Wei WANG, Somerville, MA (US); Jun LI, Cambridge, MA (US); Yugang JIA, Winchester, MA (US)

(21) Appl. No.: 15/960,926

(22) Filed: Apr. 24, 2018

Related U.S. Application Data
(60) Provisional application No. 62/490,943, filed on Apr. 27, 2017.

(51) Int. Cl. G16H 40/20; G06Q 50/22; G06Q 10/06

(52) U.S. Cl. CPC G16H 40/20; G06Q 10/1095; G06Q 10/06311; G06Q 50/22

(57) ABSTRACT

A method and an apparatus for accurately predicting and modeling patient events, such as avoidable admissions, within a healthcare network are disclosed herein. The present disclosure provides systems and methods of predicting and modeling patient events with the use of a constantly updated data set, a sliding windows format, and a random survival forest model. Further, the present disclosure provides methods and systems for accurately predicting and modeling patient events and patient flows amongst various facilities within, and outside of, the healthcare network. The present application provides systems and methods for overcoming problems associated with conventional simulation systems by providing an intuitive and concise validation procedure to tune the simulation system, particularly targeting at patient flow among the various facilities, or nodes.

### CROSS-REFERENCE TO PRIOR APPLICATIONS

This application claims the benefit of United States Provisional Application No. 62/490943, filed on Apr. 27, 2017. This application is hereby incorporated by reference herein.

### FIELD

The present application relates generally to patient flow simulations. More particularly, the present application relates to systems and methods for generating patient admissions, for generating patient flow simulations between hospitals, and for validating patient flow simulation on healthcare networks.

### BACKGROUND

Healthcare delivery entities are hospitals, institutions and/or individual practitioners that provide healthcare services to individuals. In recent years, there has been an increased focus on monitoring and improving the delivery of healthcare around the globe. Traditionally, healthcare delivery has been driven by volume, meaning that healthcare delivery entities are motivated to increase or maximize the volume of healthcare services, visits, hospitalizations and tests that they provide.

More recently, there is a growing trend in which healthcare delivery is shifting from being volume driven to being outcome or value driven. This means that healthcare delivery entities are being incentivized to provide high quality healthcare while minimizing costs, rather than simply providing the maximum volume of healthcare. One way in which healthcare delivery entities are being incentivized is by the implementation of payment systems in which healthcare delivery entities (e.g., Accountable Care Organizations (ACOs)) are paid using a pay-for-performance model.

This shift to outcome or value driven service has thus increased the importance of defining, monitoring, and measuring the quality of healthcare, namely focusing on safe, effective, patient-centered, timely, efficient, and equitable healthcare delivery. Healthcare quality measurements are used by emerging outcome or value driven payment models, for example, to benchmark performance against other providers, thereby improving transparency, accountability, and quality; reward or penalize healthcare delivery entities or services that either meet or do not meet certain quality criteria; or conform to medical, environmental, and other like standards or guidelines related to healthcare delivery.

As a result of this shift, healthcare providers have been seeking ways to intuit expected needs of patients and healthcare facilities. This is important for at least two reasons. As a first matter, being able to accurately predict the needs of patients can allow for healthcare networks to maintain facilities with sufficient bandwidth to timely treat patients without long wait times. Secondarily, healthcare provider management has been seeking the capability to predict patient visit patterns in the future. As such there is a need for accurate models that can simulate and predict patient visit patterns that can provide healthcare management the ability to redirect resources, such as staffing and medical supplies. Further, accurate models of patient flows within a network can then, for example, inform strategic operating decisions such as the creation of new facilities and clinic allocations.

This new healthcare model can raise new challenges within healthcare delivery networks. For example, such systems need to be able to analyze (1) whether their programs are on the right track to achieve internal, and external, goals and benchmarks; (2) what impact changes in the network may have; (3) what service line and/or practices the systems should focus on; and/or (4) where the systems is missing critical network coverage. Simulating patient behaviors can be an effective means to perform the aforementioned analyses, as well as others.

Simulating patient behaviors within the networks can be but one instrumental part of building a better healthcare delivery system both for the healthcare payers and consumers. Accurate simulations can enable network staff to analyze various scenarios of patient behaviors without the need for costly and time intensive observation of actual patient behavior. However, simulations and modeling of patient behaviors have been mostly restricted to the scale of a single healthcare facility, and simulation of patient behaviors on a large scale network-level is needed.

Simulations of patient flows under different scenarios may help the decision makers and stakeholders to gain insight about the system and optimize patient experience. However, simulation of a complex large scale network level can be difficult. Modeling large scale networks requires modeling patient flow among the various network nodes, i.e., various healthcare providers. This type of simulation can be instrumental to understanding patient behaviors and optimizing the intricate healthcare system. A central challenge constructing large scale network simulations is the lack of appropriate validation criteria to assess the quality of the simulated data.

Thus, there is a need for improved systems and methods that enable intuitive and concise validation procedure to tune the simulation system, particularly targeting at patient flow among a plurality of nodes. Validation procedures may generate high quality simulated patient flow, which can be valuable for strategic analysis and consulting engagements with big hospital systems.

### SUMMARY

The present disclosure provides methods and systems for accurately predicting and modeling patient events, such as avoidable admissions, within a healthcare network generally.

Further, the present disclosure provides methods and systems for accurately predicting and modeling patient events and patient flows amongst various facilities within, and outside of, the healthcare network. The present application provides systems and methods for overcoming problems associated with conventional simulation systems by providing an intuitive and concise validation procedure to tune the simulation system, particularly targeting at patient flow among the various facilities, or nodes.

Various advantages and other features of the structures and methods disclosed herein will become more readily apparent to those having ordinary skill in the art from the following detailed description of certain preferred embodiments taken in conjunction with the drawings which set forth representative embodiments of the present disclosure and wherein like reference numerals identify similar structural elements.

In an exemplary method of evaluating results of simulations in healthcare networks, the method includes reading historical data of patient visits for a plurality of first locations and a plurality of second locations from a database, constructing a historical patient flow matrix by calculating patient flow between the plurality of first locations and the plurality of second locations; constructing a plurality of simulated patient flow matrices patient flows by simulating patient flows between the plurality of first locations and the plurality of second locations based on a plurality of sets of parameter values; and determining the best set of parameter values among the plurality of sets of parameter values by comparing the historical patient flow matrix and the plurality of simulated patient flow matrices.

In some embodiments, the comparing the historical patient flow matrix and the plurality of simulated patient flow matrices step can further include calculating a distance between the historical patient flow matrix and each of the plurality of simulated patient flow matrices. The best set of parameter values can correspond to a simulated patient flow matrix having the shortest distance from the historical patient flow matrix.

In some embodiments, the parameter values can include at least one of a travel distance for a patient visit, waiting time of the plurality of locations, and reputation of the plurality of locations. Zip codes for a plurality of patients, the plurality of the locations are stored in the database. The travel distance for the patient visit can be calculated based on the zip codes for a plurality of patients and the plurality of locations. The plurality of sets of parameter values can be configurable by a user through a user interface.

In one exemplary embodiment, an apparatus for evaluating results of a simulation in healthcare networks can include a data storage unit storing historical data of patient visits for a plurality of first locations and a plurality of second locations; a patient flow matrix building unit building patient flow matrix from based on data of patient visits; a patient flow simulation unit generating simulated data of patient visits between the plurality of first locations and the plurality of second locations based on a set of parameter values; and a simulation evaluation unit. The simulation evaluation unit can read the historical data of patient visits from the data storage unit, constructing a historical patient flow matrix with the patient flow simulation unit using the historical data of patient visits, constructing a plurality of simulated patient flow matrices patient flows with the patient flow simulation unit using a plurality of simulated data of patient visits generated by the patient flow simulation unit, and determining the best set of parameter values among the plurality of sets of parameter values by comparing the historical patient flow matrix and the plurality of simulated patient flow matrices.

In some embodiments, the simulation evaluation unit can compare the historical patient flow matrix and the plurality of simulated patient flow matrices by calculating a distance between the historical patient flow matrix and each of the plurality of simulated patient flow matrices. In further embodiments, the best set of parameter values can correspond to a simulated patient flow matrix having the shortest distance from the historical patient flow matrix. Sometimes, the parameter values can include at least one of a travel distance for a patient visit, waiting time of the plurality of locations, and reputation of the plurality of locations. Further, the zip codes for a plurality of patients and the plurality of the locations can be stored in the database. Moreover, the travel distance for the patient visit can be calculated based on the zip codes for a plurality of patients and the plurality of locations. Further still, the plurality of sets of parameter values are configurable by a user through a user interface.

It should be appreciated that the present technology can be implemented and utilized in numerous ways, including without limitation as a process, an apparatus, a system, a device, a method for applications now known and later developed or a computer readable medium.

Other aspects and advantages of the invention can become apparent from the following drawings and description, all of which illustrate the principles of the invention, by way of example only.

### BRIEF DESCRIPTION OF THE DRAWINGS

The present application will be more fully understood from the following detailed description taken in conjunction with the accompanying drawings, in which:

FIG. 1 illustrates a flow chart showing a patient decision path for choosing a healthcare facility;

FIG. 2A illustrates a block diagram showing an exemplary simulation system;

FIG. 2B illustrates a block diagram showing an exemplary configuration of a simulation system;

FIG. 3 illustrates exemplary flows of patients between first locations and second locations;

FIG. 4A illustrates a flow chart showing the steps of evaluating simulation results;

FIG. 4B illustrates a flow chart showing detail steps of comparing historical result with simulated results;

FIG. 5 illustrates a block diagram showing the configuration of a simulation evaluation system;

FIG. 6 illustrates data of demand, capacity, and patient outflow at local healthcare facilities;

FIG. 7 illustrates one example of a prediction model output of patient outflow;

FIGS. 8A-8C illustrate patient allocation matrices;

FIGS. 9A-9C illustrate patient outflow matrices;

FIG. 10 illustrates model and simulation based results of the instant system; and

FIG. 11 illustrates a chart visualization model and simulation based results of the instant system.

### DESCRIPTION

Certain exemplary embodiments will now be described to provide an overall understanding of the principles of the structure, function, manufacture, and use of the systems and methods disclosed herein. One or more examples of these embodiments are illustrated in the accompanying drawings. Those skilled in the art will understand that the systems and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Further, in the present disclosure, like-numbered components of various embodiments generally have similar features when those components are of a similar nature and/or serve a similar purpose.

The present disclosure provides methods and systems for accurately predicting and modeling patient events, such as avoidable admissions, within a healthcare network generally. For example, the present disclosure may provide detailed simulations of individual patient cohorts within the network. The present disclosure provides systems and methods of predicting and modeling patient events with the use of a constantly updated data set, a sliding windows simulation, and a random survival forest model. Further, the present disclosure provides methods and systems for accurately predicting and modeling patient events and patient flows amongst various facilities within, and outside of, the healthcare network. The present application provides systems and methods for overcoming problems associated with conventional simulation systems by providing an intuitive and concise validation procedure to tune the simulation system, particularly targeting at patient flow among the various facilities, or nodes.

The present disclosure provides validation programs may generate high quality simulated patient flow, which can be valuable for bettering outcome driven healthcare, strategic analysis, and consulting engagements with big hospital systems. These programs can be implemented individually or collectively as a software suite or a software dashboard. Such a software suite can accept raw data, as discussed below, and output processed information via a console or display that is helpful to healthcare managers, doctors, nurses, and hospital administrators. The present disclosure can leverage large volumes of raw data flows from various sources within a healthcare network to continuously update and tune simulation systems. Various advantages and other features of the structures and methods disclosed herein will become more readily apparent to those having ordinary skill in the art from the following detailed description of certain preferred embodiments taken in conjunction with the drawings which set forth representative embodiments of the present disclosure and wherein like reference numerals identify similar structural elements.

In some systems, it can be beneficial to forecast future patient visits. For example, such a system can simulate patient cohort hospital visits within a healthcare network. A patient cohort can be understood as a group of patients all having generally similar medical conditions, such as congestive heart failure, within a single healthcare network. In general, the simulation system can include a database of patient features and historical visits of patients within a cohort between different healthcare facilities; a dynamic survival model; a patient choice model; and a pipe line between the two models as discussed in U.S. Patent Application No. 62/490855, entitled “METHODS AND APPARATUS FOR DYNAMIC EVENT DRIVEN SIMULATIONS,” Docket No. 2016PF01260, filed on an even date herewith, which is incorporated by reference herein in its entirety. While the systems herein are discussed with reference to patients, and healthcare networks generally, the dataflow and algorithms described herein can be applied to other event-driven networks such as communication network routing systems.

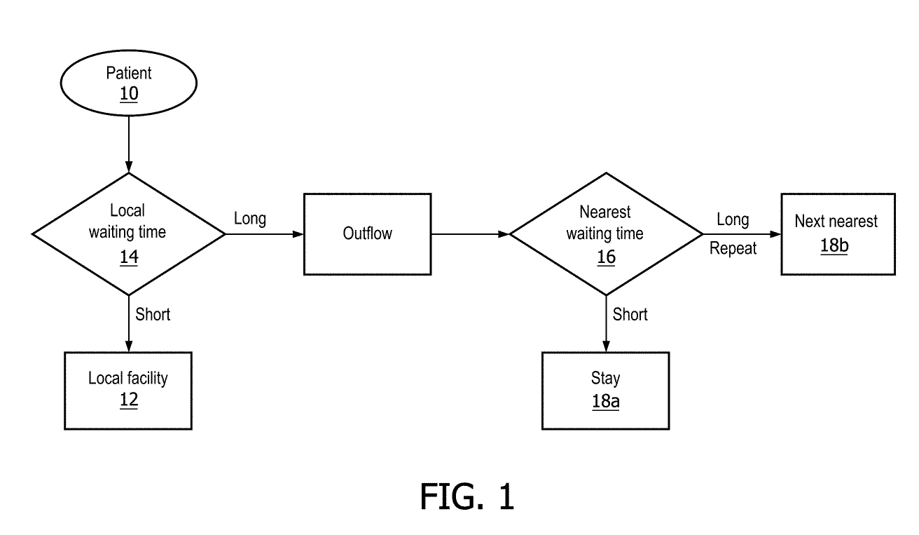

Understanding and modeling patients' behavior can be one important information source for minimizing the number patients leaving a local healthcare facility (“patient outflow”), for optimizing patient experience and life expectancy, and enhancing the overall healthcare network. A decision flow chart is shown in FIG. 1. The flow chart of FIG. 1 can represent the decisions individual patients 10 consider when a visit to a local facility 12 is required. The patient 10 can consider the local wait times 14 at the local facility, if that time is short enough they will likely decide to go to the local facility. However, if the local wait time 14 is too long, the patient will look to the wait time 16 at the next closest facility 18a, this is considered patient outflow. If the wait time at the next nearest facility 18a is acceptable the patient may stay, or alternatively can look to another facility 18b still further away. The patient 10 may balance the length of the wait time with the distance from the facility. Additionally, the patient may consider the reputation of a given facility as yet another factor in the decision making process. Historical behavior models can provide an initial guide for future modeling and simulation of patient flows within the network. With modern Centers for Medicare and Medicaid Services (CMS) and established Accountable Care Organizations (ACOs), there is a wealth of data being collected in digital form for each patient visit. For example, this data can include vast amounts of information pertaining to the networks performance down to individual patient movements through the systems. The historical data can include information regarding the local waiting time at a given medical facility and when patients prefer their local facility. The historical data can indicate, for example, when there is a long wait time a patient will go out of current municipality and go to the nearest one. Alternatively, if the wait time in a given municipality is short, a patient is more likely to stay and receive medical treatment there. Being able to predict this behavior amongst individual patients or patient cohorts can be helpful in balancing network resources and improving patient outcomes.

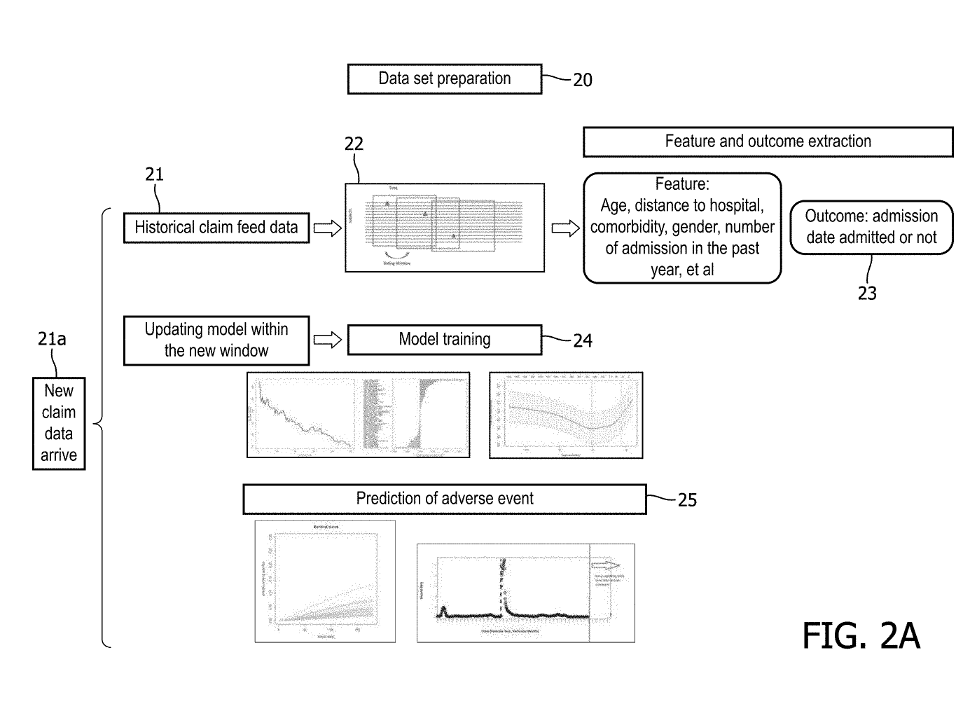

As shown in FIG. 2, as a result of the collaboration between CMS and ACOS, the ACOs may request to receive historical monthly data feed files 21 from their beneficiaries who receive primary care services and have not declined to share their information. Such claim data can contain health condition and visiting episode information for the patient, or beneficiary, which is essential in building a prediction model, that is can be cost-effective to collect and low-latency to obtain. In one example, certain ACOs may serve over 60,000 Medicare beneficiaries. Data for the 60,000 beneficiaries can be collected from the monthly data files called “Accountable Care Organization-Operational System (ACO-OS) Claim and Claim Line Feed (CCLF)”. In one example, a data stream random survival forest (DSRSF) model, as described in the aforementioned U.S. Patent Application 62/490,855, can be used to predict future readmissions of patients in a particular cohort to the healthcare network.

The data stream random survival forests model offers a powerful and efficient way to do risk stratification of beneficiaries using data streams in medical area such as monthly updated claim data released from CMS. It can be easily extended to handle a large amount of data and deployed for the practical use. Practical use can include future investments in healthcare facilities and other durable medical equipment across an entire large scale healthcare network.

As shown in FIG. 2B, data is inputted into the system 50 that can perform feature engineering 23, then formatted as the sliding windows 22, and the survival model 30 to predict the likelihood of an avoidable admission (or other adverse event). Then the adverse event simulation 25, 30 can be implemented based on the risk profile 30. As noted above, once an adverse event occurs, i.e., the need for a hospital visit arises. At such a time the beneficiary, or patient, then makes a choice 52 regarding which hospital to go for the healthcare service. Since the DSRSF model concerns a particular patient cohort, such as congestive heart failure (CHF) patients, it is reasonable to assume that the medical needs amongst the individuals within the cohort are not very different. Therefore, the choice of which hospital 62a-f to attend can be simplified to depend on three factors, hospital reputation, distance to hospital from the patients' locations, residences, or nodes 60a-f, and waiting time at the hospital 62a-f. Mathematically, this can be expressed as a cost function of patient choosing each of the hospital, and the patient would choose the hospital with the lowest cost. Each of the factors, or variables, can be weighted by means of a calculated coefficient to ensure that the model is accurate. The coefficients, or parameters, of the cost function can be fine-tuned by doing a grid-search and then evaluating the resemblance of the simulated data and the historical data, as discussed further below.

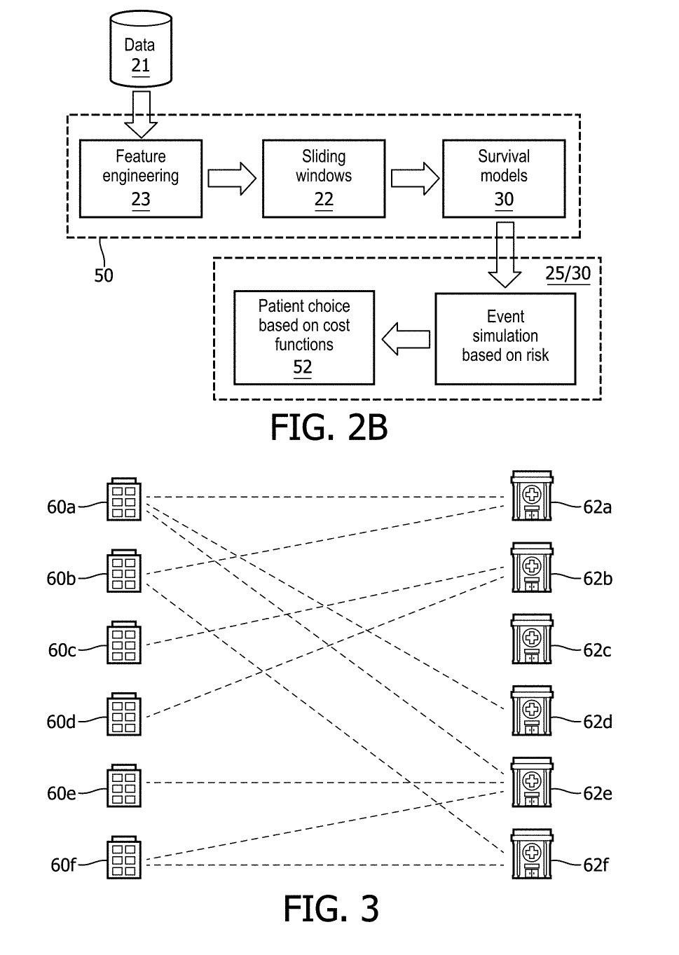

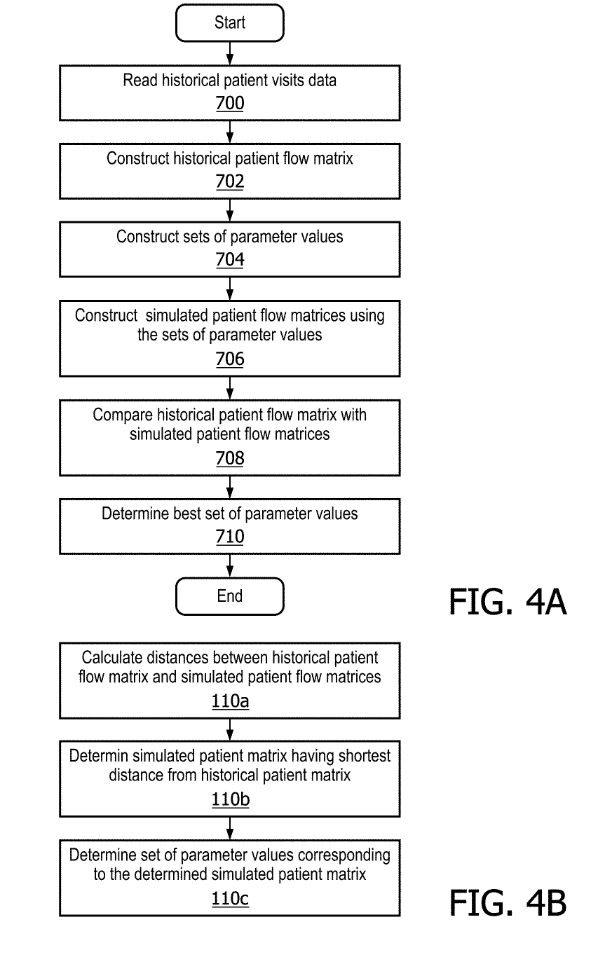

As shown in FIGS. 3 and 4A-4B, this process can begin by reading the historical patient visit data 700, including the patient's location or node (zip code) 60a-f and the location or node of the medical facility 62a-f that the patient ultimately visited. The system can then construct a historical patient flow matrix to represent the historical data 702. Initial parameters, or coefficients, for the cost function can be determined by the system 704, then the system can construct a simulated patient flow matrix using the historical data and the aforementioned coefficients 706. The system can then validate the cost function, and the associated coefficients, by comparing the historical patient flow matrix with the simulated patient flow matrix, as discussed further below 708. If the two matrices are too divergent, the parameters can then be adjusted to ensure that the implemented cost function is accurate 710. As shown in FIG. 4B, the parameters can be adjusted by calculating the distances between historical patient flow matrices and the simulated flow matrices 710a, and determining which simulated patient matrices have the shortest distance from the historical patient matrix 710b. The system can then determine a new set of parameter values corresponding to the simulated patient matrix that has the shortest distances form the historical patient matrix 710c. Further, a user can manually adjust the parameters as required to further fine tune the system if required.

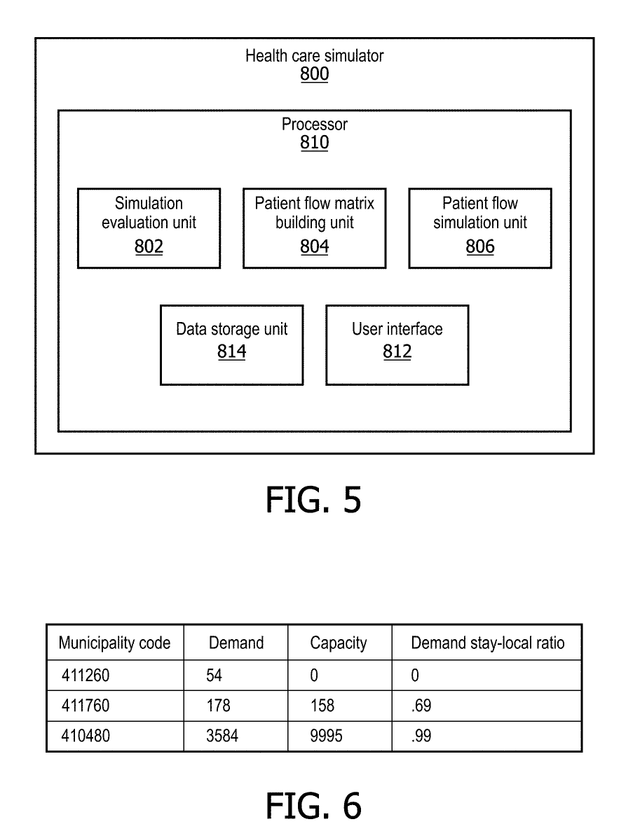

The output of the simulator 800, as shown in FIG. 5, for example the simulation evaluation unit 802, the matrix construction unit 804, and patient flow simulation unit 806, as performed by a digital data processor 810 can be displayed on a dashboard, or user interface, 812 that is viewable by hospital staff and healthcare network administrators. This data stored in a data storage unit 814, as noted above, can be useful to predict needs across the various healthcare facilities within a single network. The aforementioned coefficients can be calculated and tuned on the back end of the system, such that the end user viewing the dash board can make determinations with confidence that the data being shown is as accurate as possible.

For each individual patient in the cohort, a dynamic survival model can be applied to predict the likelihood of predictable admission events with the occurring time information. At each actual occurrence of an event, the patient makes a choice of which hospital to seek medical services according to the cost functions. The choice is normally dynamic because it involves the current system status, i.e., the waiting time (queue length) at each hospital under considerations. The system can be run for a fixed amount of time, and a complete picture of the patient cohort hospital usage can be created. For statistical validity, the simulation system can be rerun multiple times and confidence intervals of key system performance indicators, such as throughput and peak patient load at each hospital, can be obtained.

As noted above, large networks in modern healthcare delivery can include integrated delivery networks (IDN); accountable care organizations (ACO); and/or public health systems. Understanding and modeling patients' behaviors across multiple episodes of care are crucial to achieve the goals in large networks. Those goals can include minimizing ACO patient leakage, optimizing patients' experience (e.g., waiting time or travel distance), and minimizing overall healthcare expenditures.

Understanding and modeling patients' future behaviors is important for minimizing patient outflow and for optimizing patients experience and so on. As such, the behavior model can directly guide modeling and simulation used for planning further expenditures and expansions. For example, as discussed above, if a patient requires medical attention, they will likely proceed to the local facility in their municipality. Further, if the waiting time at the local facility is short, the patient will prefer the local facility. However, if the wait time to be seen is too long, the patient will look to leave their local municipality and go to a second facility in the next nearest municipality. If the waiting time at the second facility is short, the patient will stay. Alternatively, if the wait time at the second facility is long, the patient will go to a third municipality, and so on. This procedure could be represented by the flowchart illustrated in FIG. 1.

Based on the data format and the patient behavior model, the instant embodiment can use two different methods to solve for optimal allocation of resources, such as Mill machines. The two methods can include a top-down model based optimization procedure and a bottom-up simulation method. Each model can serve as corroboration for the other to validate the output from the system as a whole. Individually, both methods have advantages and disadvantages that serve to balance the other to provide consistent and interpretable results.

The top-down model, or optimization framework, can fit aggregate-level (municipality) models. Advantageously, top-down model can be concise and have optimal allocations that are easily derivable. However, top-down models can have low resolution and might be too rough and include ecological fallacies. Alternatively, a bottom-up agent-based simulation can advantageously recreate the whole network and can be empirically grounded. Some draw backs to the bottom-up agent-based simulation can be that they are hard to tune and optimal allocations are not easily derivable. Further, the recommended allocation can be hard to validate since a “what-if” scenario analysis is often about counterfactual or the future variables. For example, patients' behaviors may change if the healthcare system adds capacity by adding a new facility or by adding capacity at an existing location. Starting from diametric perspectives, simulation- and optimization-based approaches can be used to validate and corroborate each other.

When dealing with planning of expenditures, for example new MRI machines, management of large healthcare networks may take the followings into account: (1) Regional imbalance; (2) efficiency vs equity; (3) patient outflow; and/or (4) procedure costs. In one embodiment, a data-driven approach can be designed. As discussed above, the system can look at historical data from the network over a specified period. For example, the system can look at 3 million exams within a particular network over 2 years. Those exams can include MRI exams (0.1 million), Tomography exams (0.6 million), Ultrasound exams (1.43 million) and X-ray exams (0.8 million). The network itself can be divided into a plurality of municipalities and the system can locate patients and facilities to municipality level resolution. In the instant example, the network can include 399 municipalities.

Top-down Aggregate-Level-Based Optimization Framework

To start the model-based approach using aggregate exam-level data the data, into one usable format. For example, the data can be sorted by municipalities, as shown in FIG. 6. For each municipality data is shown corresponding to the number of patients from a particular municipality, i.e. the demand, the total number of patients treated in the municipality, i.e., capacity, and the proportion of local people that chose the local hospital, i.e. the demand stay-local ratio.

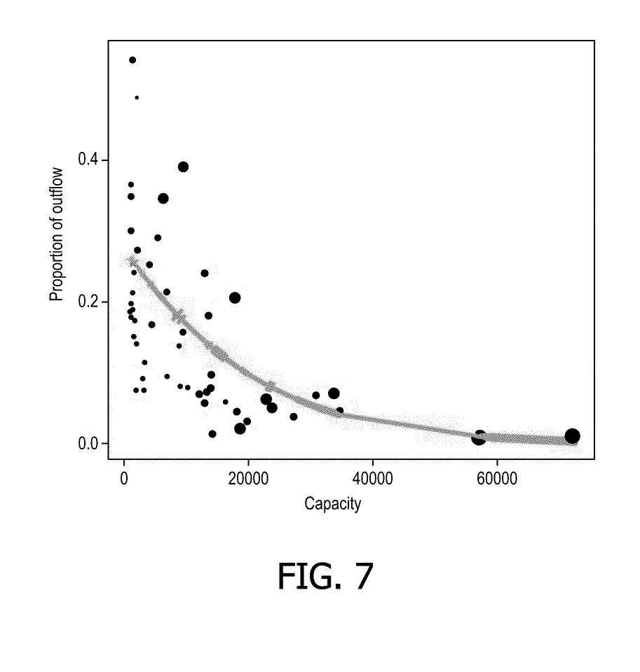

Since the outcome of whether the patient chooses to go to a local hospital is binary in nature, logistic regression can be a natural modeling choice, as illustrated in FIG. 7. Alternatively, any modeling technique targeted at binary outcomes can be used. As shown in FIG. 7, there is a strong relationship between the total capacity and proportion of outflow at a municipality level. To validate the model, the average area under the ROC curve is computed using, for example a five-fold cross-validation. Alternatively, other validation methods may be employed. In the illustrated example, the validation obtains a result of 0.96 which is a significant score that indicates a municipality's capacity can be a good predictor for patient outflow status. This method can be useful for predicting within a static system, or network, future patient flow. However, healthcare networks are constantly enlarging and shifting, therefore there is an added need to optimize the framework to account for additions in the capacity of a network.

For example, assuming that a healthcare network adds new capacity that is equal to 10% of the original total capacity, in that case, the total number of outflow patients can be calculated using sequential quadratic programming according to the following equations:

$${min}_{Δ {C}_{K}} {\sum }_{k} {D}_{k} \times f ( {C}_{K} + Δ {C}_{K} )$$

$$s . t . {\sum }_{k} Δ {C}_{k} = fixed amount$$

where Dₖ is the demand of municipality k; Cₖ is the current capacity at municipality k; f is the estimated preference function; and ΔCₖ is the new capacity to be added to municipality k. The total number of outflow patients will reduce by 2.3% if the new capacity that is added is proportional to original capacity distribution, so-called the baseline. However, if the new capacity that is added is calculated according to optimized allocation, the total number of outflow will reduce by 5%. This is compared with the baseline method, the reduction of patient flow is nearly doubled by using optimized allocation.

The Bottom-up Agent-Based Simulation Model

Agent-based simulation models can be effective to simulate the actions of autonomous patients with a view to assess their effects on the healthcare system as a whole. In such a model, two terms can be used in the simulation framework, the patient arrival rate and the service rate. The patient arrival rate can be proportional to the demand in a given municipality so that a newly simulated patient has higher probability from a municipality with higher demand. The service rate is proportional to municipality capacity, where a high service rate indicates the queue is shortening fast. As such a patient is simulated by the model, according to demand information. There are then two factors that will affect the patients decision as to which municipality they will visit to receive medical care: the distance to the facility and the waiting time at that given facility. These two factors are variables in the resulting patient cost function.

One patient cost function, as discussed above, that is associated with the patient choosing a particular municipality k can be resented as follows:

cost=w*log(distance_to_k)+waiting_time_k

where w is the coefficient that connects distance and waiting time measures. A log scale of distance may be used, due to the better output performance of the formula. The formula additional uses queue, or line, length to describe the waiting time. Once a particular patient has made his decision to go to a municipality, this will increase the queue length by 1 at that municipality. Each of the queues can shorten proportional to the municipality capacities as a function of time. The coefficient w thus needs to be tuned to optimize the cost function.

In one embodiment, the coefficient w can be tuned with the use of a grid-search procedure. The grid-search procedure can tune the coefficient w, using Outflow Bias, Municipality Throughput and Patient Flow matrix as criteria. The outflow bias can compute difference between simulated outflow and true outflow form each municipality. The municipality throughput criteria can compute difference between simulate patient allocation vector and true patient allocation vector. The patient flow matrix criteria can compute a patient flow matrix is a 399-by-399 matrix which i, j the entry shows how many patients travel from municipality i to municipality j, and can compute the difference between the patient flow matrix from simulation and that from true data. In some embodiments, if the outflow bias is controlled to be nearly 0, the other two criteria can also achieve respective lowest values. Alternatively, any cross-validation technique can be used to tune the coefficient w. In some embodiments, the end users of the models will have access to modify the coefficients

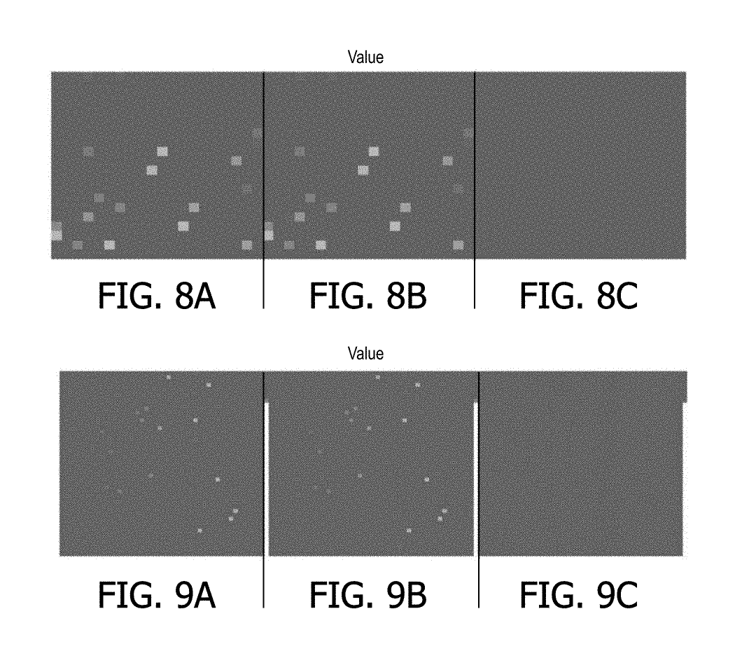

For example, as illustrated in FIGS. 8A, 8B, 8C, the differences between true and simulated patient allocation vectors are shown. Where FIG. 8A illustrates a matrix of the true patient allocations, FIG. 8B illustrates a matrix of the simulated patient allocations, and FIG. 8C illustrates the difference between the true and simulated patient allocations. Further, as illustrated in FIGS. 9A, 9B, 9C, the differences between true and simulated patient flow vectors are shown. Where FIG. 9A illustrates a matrix of the true patient flow, FIG. 9B illustrates a matrix of the simulated patient flow, and FIG. 9C illustrates the difference between the true and simulated patient flows. In all of FIGS. 8A-C and 9A-C, the plots show the accord between simulated and true data.

Validation of the Recommended Allocation

As discussed herein, the validation of “what-if” or unknown future event scenario analysis is inherently difficult. Therefore, it may be necessary to compare the decreases in outflow patients for both optimization and simulation under two scenarios. Those two scenarios can be the baseline proportional to capacity and the optimization-induced allocation.

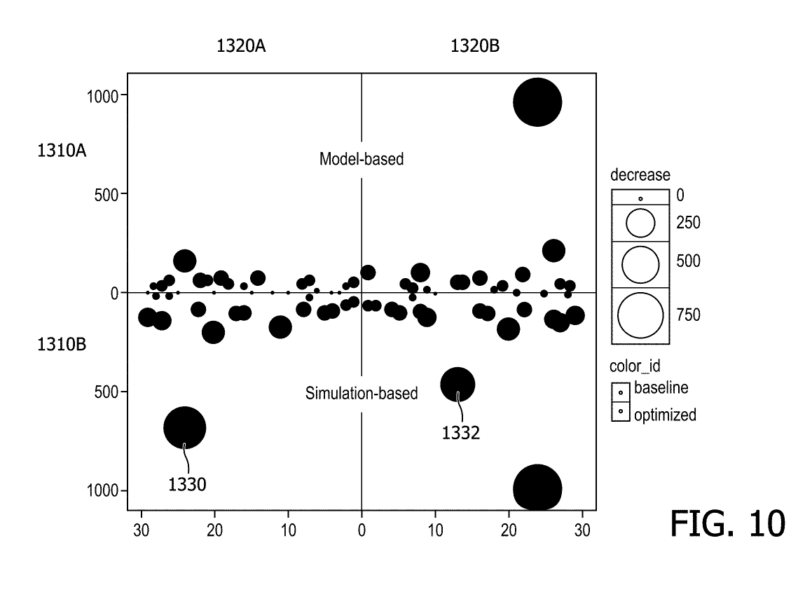

As illustrated in FIG. 10, the upper half 1310A can show the model-based results while the lower half 1310B can show the simulation-based result. The bubbles in the right half 1320A of the chart can represent the optimized decreases and the bubbles on the left half 1320B of the chart can represent the baseline decrease. In the illustrated example, there are only one outlier each 1330, 1332 in the simulation based bubbles. This can illustrate that the model-based method and simulation-based method support the findings of the other.

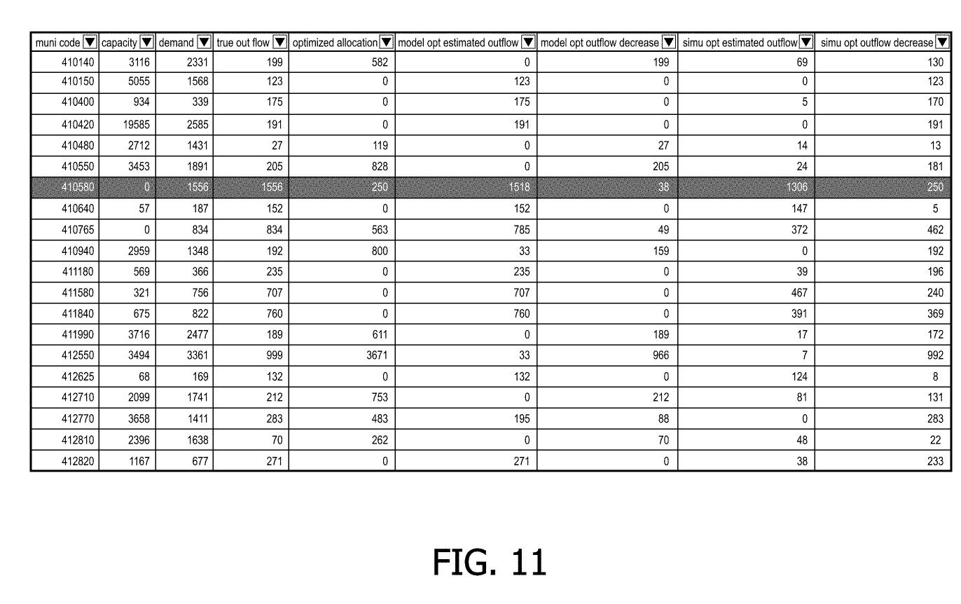

In the illustrated example, the outlier bubbles 1330, 1332 may represent how the model underestimated the decrease. Therefore, it would be acceptable to understand that the simulation results would seem more reliable. This may be because, the range of MRI's capacity and its demand are both about 0-20000, as shown in FIG. 11. The allocation of 250 new MRIs is relatively small when comparing with 20000, which leads to the underestimation of the decreased proportion. What's more, the demand for that municipality is also not large when comparing with 20000. The two factors lead to an underestimation of the decrease.

In summary, the model based method can, in general, calculate the optimal allocation more easily, however the simulation result is more scale-robust and therefor ultimately more reliable. Thus, business decision makers within the healthcare network are able to make long term planning and allocation decision for the healthcare network. Further, the business decision makers are able to determine if reallocation of resources within the network is needed, thereby optimizing the healthcare network. Therefore, each of the aforementioned goals, minimizing ACO patient leakage, optimizing patients' experience (e.g., waiting time or travel distance), and minimizing overall healthcare expenditures, can be achieved.

Each of the aforementioned systems and models can be applicable in a healthcare network, however, it is contemplated that the modeling and prediction methods disclosed herein can be applicable in a variety of other systems. Moreover, each of the prediction models and algorithms can be part of a software suite or be used individually. The models and algorithms can be processed in the cloud on a remote digital data processor that outputs data, or reports, to end users via a dashboard that is visually depicted as a graphical user interface (GUI). The data or reports can be printed by means of a printer, displayed on a monitor, emailed, or otherwise delivered to end users. The dashboard can be merely an output such that an end user does not have the ability to modify any coefficients, assumptions, or data sets inputted into the system.

While the foregoing description has been directed to specific embodiments, it will be apparent that other variations and modifications may be made to the described embodiments, with the attainment of some or all of their advantages. Accordingly this description is to be taken only by way of example and not to otherwise limit the scope of the embodiments herein. Finally, all publications and references cited herein are expressly incorporated by reference in their entirety.

What is claimed is:

1. A method for evaluating results of simulations in healthcare networks, the method comprising:
reading historical data of patient visits for a plurality of first locations and a plurality of second locations from a database;
constructing a historical patient flow matrix by calculating patient flow between the plurality of first locations and the plurality of second locations;
constructing a plurality of simulated patient flow matrices patient flows by simulating patient flows between the plurality of first locations and the plurality of second locations based on a plurality of sets of parameter values; and
determining the best set of parameter values among the plurality of sets of parameter values by comparing the historical patient flow matrix and the plurality of simulated patient flow matrices.

2. The method of claim 1, wherein the comparing the historical patient flow matrix and the plurality of simulated patient flow matrices further comprises calculating a distance between the historical patient flow matrix and each of the plurality of simulated patient flow matrices.

3. The method of claim 2, wherein the best set of parameter values corresponds to a simulated patient flow matrix having the shortest distance from the historical patient flow matrix.

4. The method of claim 1, wherein the parameter values include at least one of a travel distance for a patient visit, waiting time of the plurality of locations, and reputation of the plurality of locations.

5. The method of claim 4, wherein zip codes for a plurality of patients, the plurality of the locations are stored in the database.

6. The method of claim 5, wherein the travel distance for the patient visit is calculated based on the zip codes for a plurality of patients and the plurality of locations.

7. The method of claim 1, wherein the plurality of sets of parameter values are configurable by a user through a user interface.

8. An apparatus for evaluating result of simulation in healthcare networks, the apparatus comprising:
a data storage unit storing historical data of patient visits for a plurality of first locations and a plurality of second locations;
a patient flow matrix building unit building patient flow matrix from based on data of patient visits;
a patient flow simulation unit generating simulated data of patient visits between the plurality of first locations and the plurality of second locations based on a set of parameter values; and
a simulation evaluation unit reading the historical data of patient visits from the data storage unit, constructing a historical patient flow matrix with the patient flow simulation unit using the historical data of patient visits, constructing a plurality of simulated patient flow matrices patient flows with the patient flow simulation unit using a plurality of simulated data of patient visits generated by the patient flow simulation unit, and determining the best set of parameter values among the plurality of sets of parameter values by comparing the historical patient flow matrix and the plurality of simulated patient flow matrices.

9. The apparatus of claim 8, wherein the simulation evaluation unit compares the historical patient flow matrix and the plurality of simulated patient flow matrices by calculating a distance between the historical patient flow matrix and each of the plurality of simulated patient flow matrices.

10. The apparatus of claim 9, wherein the best set of parameter values corresponds to a simulated patient flow matrix having the shortest distance from the historical patient flow matrix.

11. The apparatus of claim 8, wherein the parameter values include at least one of a travel distance for a patient visit, waiting time of the plurality of locations, and reputation of the plurality of locations.

12. The apparatus of claim 11, wherein zip codes for a plurality of patients, the plurality of the locations are stored in the database.

13. The apparatus of claim 12, wherein the travel distance for the patient visit is calculated based on the zip codes for a plurality of patients and the plurality of locations.

14. The apparatus of claim 8, wherein the plurality of sets of parameter values are configurable by a user through a user interface.
